(19) United States
(12) Patent Application Publication
(10) Pub. No.: US 2023/0414145 A1
(43) Pub. Date: Dec. 28, 2023

(54) ELECTRODES HAVING DRY ADHESIVE SECTIONS, WEARABLE DEVICES INCLUDING SUCH ELECTRODES, AND METHOD OF MAKING AND USING SUCH ELECTRODES

(71) Applicant: NextSense, Inc., Mountain View, CA (US)

(72) Inventors: Robert DITTMER, Hanau (DE); Leonard STOICA, Hanau (DE); Jeffrey HENDRICKS, Mountain View, CA (US)

(21) Appl. No.: 18/253,354

(22) PCT Filed: Nov. 16, 2021
(86) PCT No.: PCT/US21/72443

§ 371 Date: May 17, 2023

Related U.S. Application Data
(60) Provisional application No. 63/114,697, filed on Nov. 17, 2020.

(51) Int. Cl. A61B 5/257; A61B 5/268; A61B 5/256; A61B 5/00

(52) U.S. Cl. CPC A61B 5/257; A61B 5/268; A61B 5/6817; A61B 5/6804; A61B 5/256

(57) ABSTRACT

An electrode comprising a top layer comprising at least one conductive polymer section and at least one dry adhesive section, wherein the at least one dry adhesive section comprises a plurality of fibrillar microstructures; at least one middle layer comprising a non-conductive polymer; and a bottom layer comprising a conductive polymer, wherein the conductive polymer of the bottom layer is in electrical communication with the conductive polymer of the top layer through the at least one middle layer.

### CROSS-REFERENCE TO RELATED APPLICATION(S)

This application claims priority to U.S. Provisional Application No. 63/114,697, filed on Nov. 17, 2020, the disclosure of which is hereby incorporated by reference herein in its entirety.

### FIELD OF THE INVENTION

The present disclosure relates to a dry adhesive electrode which can adhere to tissue for diagnosing, monitoring and treating patients. The present disclosure also relates to medical devices, such as wearable devices, comprising dry adhesive electrodes.

### BACKGROUND

Electrodes used in biomedical applications typically include a “wet” adhesive material, such as a biocompatible glue that is tacky, to adhere the electrode to the body of a patient. Surface electrodes in biomedical applications, such as electrocardiography or electroencephalography, often use hydrogels as a wet adhesive. Such electrodes may not adhere well to skin and over time they often require reapplication of the adhesive and/or cause skin irritation during long term use.

Smart wearable devices are items of clothing or other items (e.g., earbuds, headsets, headgear, cardiac monitors, etc.) worn by a user which incorporate electrodes and any necessary wiring harness within the wearable item for continuous monitoring and/or treatment of a person while the item is worn. Electrodes that require adhesives are often impractical for use in smart wearables that are worn for extended periods of time because conventional adhesives can irritate the skin of a user and often become less effective over time. For example, sweat from exercise and/or water from bathing can reduce the effectiveness of conventional adhesives. In many situations it is also helpful if an electrode is relatively flexible to adhere to the contours of the body and allow for free movement. Accordingly, there is a need for improving the adhesion of electrodes to the skin of a user for monitoring physiological parameters and delivering electromagnetic therapies.

### SUMMARY

The electrodes described in the present disclosure provide dry-adhesive electrodes that can be worn for extended periods without undue skin irritation or loss of adhesiveness. In one aspect, disclosed herein is an electrode comprising: a top layer comprising at least one conductive polymer section and at least one dry adhesive section, wherein the at least one dry adhesive section comprises a plurality of fibrillar microstructures; at least one middle layer comprising a non-conductive material; and a bottom layer comprising a conductive material, wherein the conductive material of the bottom layer is in electrical communication with the conductive polymer of the top layer through the at least one middle layer.

In another aspect, disclosed herein is a medical device comprising: a current delivery or receiving device and at least one electrode. The electrode comprises: a top layer comprising at least one conductive polymer section and at least one dry adhesive section, wherein the at least one dry adhesive section comprises a plurality of fibrillar microstructures; at least one middle layer comprising a non-conductive material; and a bottom layer comprising a conductive material, wherein the conductive material of the bottom layer is in electrical communication with the conductive polymer of the top layer through the middle layer. The medical device also includes at least one connector element connecting the at least one electrode to the current delivery or receiving device.

In yet another aspect, disclosed herein is a wearable garment comprising a substrate and at least one electrode. The electrode comprises: a top layer comprising at least one conductive polymer section and at least one dry adhesive section, wherein the at least one dry adhesive section comprises a plurality of fibrillar microstructures; at least one middle layer comprising a non-conductive material; and a bottom layer comprising a conductive material, wherein the conductive material of the bottom layer is in electrical communication with the conductive polymer of the top layer through the at least one middle layer.

### BRIEF DESCRIPTION OF SEVERAL VIEWS OF THE DRAWINGS

The following drawings are illustrative of embodiments of the present invention and therefore do not limit the scope of the invention. The drawings are not to scale (unless so stated) and are intended for use in conjunction with the explanations in the following detailed description. Embodiments will hereinafter be described in conjunction with the appended drawings wherein like numerals/letters denote like elements.

FIG. 1 is a cross section view of an embodiment of an electrode disclosed herein;

FIG. 2 is a top view of the embodiment shown in FIG. 1;

FIG. 3A illustrates a perspective view of exemplary microstructures on a surface of a dry adhesive as disclosed herein in an unloaded state;

FIG. 3B illustrates a perspective view of exemplary microstructures on a surface of a dry adhesive as disclosed herein in a loaded state;

FIG. 4 is a cross section view of exemplary microstructures on a surface of a dry adhesive as disclosed herein;

FIG. 5 is a cross section view of another embodiment of an electrode disclosed herein; and

FIG. 6 is a top view of the embodiment shown in FIG. 5.

### DETAILED DESCRIPTION

The following detailed description is exemplary in nature and is not intended to limit the scope, applicability, or configuration of the invention in any way. Rather, the following description provides practical examples, and those skilled in the art will recognize that some of the examples may have suitable alternatives. Examples of construction methods, materials, dimensions and fabrication processes are provided for select elements, and other elements may employ material known by those skilled in the art.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The use of the term “comprising” in the specification and the claims includes the narrower language of “consisting essentially of” and “consisting of.”

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

Wet adhesion can be described as adhering two objects through the use of a compound or substance, such as glue or paste. Electrostatic adhesion can be described as adhering two objects together via electrical charges. Electrostatic adhesion typically incorporates electrodes having opposing polarity, from which the objects will be attracted to each other. Dry adhesion can be described as adhering two objects without the use of any liquid or surface tension, but rather, through van der Waals forces. Dry adhesives are inspired by the fibrillar structures found on geckos and some spiders which rely mainly on van der Waals forces for adhesion.

Biomedical electrodes (referred to herein as simply “electrodes”) may be used for defibrillating, pacing, cardioversion, monitoring the activity of a subject's heart, and/or other neural sensing or neuromodulation applications (e.g., peripheral or spinal neural structures). Such applications can deliver therapy, such as TENS and tVNS, and/or sense neural activity. The electrodes disclosed herein are suitable for use on human subjects or patients, although use on non-human subjects is also contemplated. Examples of electrodes as disclosed herein can be coupled with power sources and control logic to deliver electrical energy to a subject, to determine the timing, levels, and history of applied energy, and to process monitored or detected data for analysis by, for example, a health care provider. Examples of electrodes as disclosed herein may be located proximate to the subject, for example, attached, connected, or coupled to the subject, at an anterior, posterior, lateral, or other location on the subject. For example, electrodes disclosed herein can be attached to the subject's chest, back, side, head (e.g., ear), abdomen, torso, thorax, or legs. In some examples, the electrodes disclosed are configured to be attached to the subject proximate to the subject's heart.

In various instances, it may be desirable to provide non-invasive externally placed electrodes for an extended period of time for defibrillation, pacing, and/or monitoring the heart of a subject, for example, while the subject is recovering from a heart attack, surgery, or other injury to the heart, while awaiting a heart transplant, or to monitor and/or protect a subject at risk of syncope. In other applications, non-invasive external electrodes may be used for sensing neural activity or delivering electrical energy to neural structures (e.g., in-ear electrodes for sensing or modulating auricular neural activity). In some prior art, externally attached biomedical electrodes may cause skin irritation at the point of attachment within a relatively short period of time ranging, for example, from about a few hours to about a few days. Extended-wear electrodes in accordance with examples of the present disclosure are constructed of materials, for example, adhesive films and electrically conducting materials, which can reduce skin irritation and/or extend the time that the electrodes may be comfortably attached to the skin of a subject.

In some examples, extended-wear electrodes in accordance with the present disclosure may be worn continuously by a subject for a time period in excess of, for example, three days, for a week or more, or for up to about two weeks or more without the subject experiencing significant skin irritation due to the attachment of the electrode to the skin of the subject. As used herein, the terms “long-term wear” or “extended-wear” refer to continuous or substantially continuous contact of an electrode with the skin of a subject for a time period in excess of, for example, three days, for a week or more, or for up to about two weeks or more. The extended-wear electrodes disclosed herein can be used for defibrillating, monitoring and/or pacing the heart of a subject, as well as other applications directed to sensing and modulating other neural activity (e.g., peripheral and/or spinal). Examples of the electrodes disclosed herein are preferably compliant with the ANSI/AAMI DF80:2003 medical electrical equipment standard for the safety of cardiac defibrillators.

In some systems, a defibrillator, TCP, or other device capable of delivering therapeutic electrical current or sensing neural activity may be incorporated into a wearable device in which case such a device can continuously monitor the physiologic status of the patient over an extended period of time of hours, days, and even months. In such cases, the wearable therapeutic system can be configured to be light and comfortable enough for a patient to be able to sleep, walk, and engage in all the normal daily activities. In particular, the current-carrying electrodes for delivery of the therapeutic electrical current may be configured such that electrical contact be made between the conductive element of the electrode and the user's skin at the time of delivery of the therapeutic current or sensing neural activity, while at the same time be wearable for extended periods without damage to the patient's skin.

In some systems, an EEG or other device capable of receiving an electrical current may be incorporated into a wearable device in which case such a device can continuously monitor the physiologic status of the patient over an extended period of time of hours, days, and even months. In such cases, the wearable therapeutic system can be configured to be light and comfortable enough for a patient to be able to sleep, walk, and engage in all the normal daily activities. In particular, the current-receiving electrodes for an electrical signal may be configured such that electrical contact be made between the conductive element of the electrode and the patient's skin at the time of receiving the current, while at the same time be wearable for extended periods without damage to the patient's skin.

Dry adhesives which do not require a glue, paste, or other adhesives can adhere to and release from contact surfaces without leaving residue on a surface with minimal contamination, allowing for repeated uses and longer lifetimes. Examples of dry adhesives include artificial fibrillar microstructures which have been shown to mimic the dry adhesive capabilities of micro-scale setae on the toes of gecko lizards. Such individual fibrillar microstructures can be configured to conform to a surface to improve real contact area and thereby increase attractive forces (e.g., intermolecular van der Waals forces) between the individual fibers and the contact surface. However, existing conductive fibrillar microstructures made of conductive particles and silicone are generally ineffective adhesives. Moreover, only a fraction of the overall area of such dry adhesive contacts the skin so much of the potential electrical contact area is wasted. Contact impedance is also affected by the amount of pressure applied during contact. Several aspects of the present disclosure overcome the shortcomings of wet adhesives and existing dry adhesives to provide a dry-adhesive electrode that is flexible, adherent, and that has good contact impedance for applications such as medical devices, which may include smart wearables. Extended-wear electrodes in accordance with examples of the present disclosure may provide one or more advantages over prior art electrodes, for example the ability to wear the electrodes for an extended period of time may reduce the number of electrodes consumed over a given period of time, reducing the cost associated with replacing electrodes which are not suitable for use in extended-wear scenarios, for example, for time periods greater than about a week.

Discomfort of a subject associated with wearing the electrodes may be decreased due to a reduction in skin irritation caused by the extended-wear electrodes as compared to conventional electrodes. Discomfort associated with wearing the electrodes may also be decreased due to a reduction in the number of times which an extended-wear electrode may need to be removed from the skin of the subject or repositioned, resulting in possible damage to the underlying skin, as compared to conventional electrodes. Further, accuracy of monitoring of the heart of a subject may be facilitated by the use of extended-wear electrodes by keeping the monitoring electrodes in the same position rather than replacing them and mounting them in potentially different positions as may occur with electrodes which should be replaced frequently or repositioned due to the occurrence of skin irritation.

In an embodiment, disclosed herein is an electrode comprising a top layer comprising at least one conductive polymer section and at least one dry adhesive section. The dry adhesive section can comprise a plurality of fibrillar microstructures. The electrode can further include at least one middle layer comprising a non-conductive material and a bottom layer comprising a conductive material. The conductive material of the bottom layer is in electrical communication with the conductive polymer of the top layer through the middle layer.

Several embodiments of electrodes described of the present disclosure can include a lead for conducting an electrical current, a metal electrode, a backing pad and a layer for electrical contact with the skin surrounded by a foam pad. The electrode is commonly attached to an electro-medical apparatus to monitor or treat a patient although wireless means of monitoring electrode readings are also available for certain circumstances.

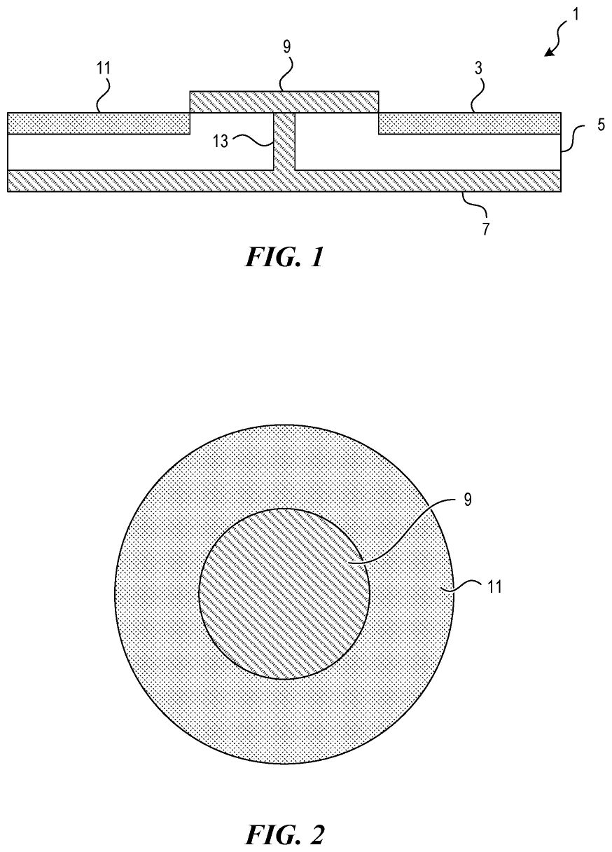

FIG. 1 and FIG. 2 show embodiments of an electrode 1 in accordance with the present technology. The electrode 1 has a top layer 3, at least one middle layer 5, and a bottom layer 7. The top layer 3 is configured to contact the skin or other tissue of a user to deliver electrical current to the user and/or sense electrical properties of the user. The top layer 3 comprises at least one conductive polymer section 9 and at least one dry adhesive section 11, and the dry adhesive section 11 can comprise a plurality of fibrillar microstructures (not shown). In FIG. 1, the conductive polymer section 9 is shown as being slightly raised relative to the dry adhesive section 11. In this embodiment, each of the conductive polymer section 9 and the dry adhesive section 11 is part of the top layer 3 even though one section may be slightly raised relative to the other. In another embodiment, the conductive polymer section 9 is substantially in the same plane relative to the dry adhesive section 11.

The conductive polymer section 9 comprises at least one conductive polymer, which functions to conduct an electric current from and/or to a person in contact with the conductive polymer. The conductive polymer is preferably a biocompatible conductive polymer that is electrically conductive and mechanically stable over one or more periods of wear. Examples of materials used for conductive polymers include polythiophene, Nafion, and polyethyldioxythiophene (PEDOT). Other classes of conductive polymers include polyacetylenes, polythiophenes (PT), and polyanilines. Conductive polymers may also include EHPT (poly(3-(2-ethylhexyl)thiophene), ionomers (e.g., NAFION®), poly(3,4 ethylene dioxythiophene) (PEDOT) and PEDOT polystyrene sulfonate (PSS/PEDOT), and mixtures thereof. The conductive polymers are biocompatible (e.g., the polymers are not toxic or injurious to living tissue). In some embodiments, PEDOT/PSS is a particularly useful conductive polymer.

In some embodiments, dopants may be added to the conductive polymer section 9 to enhance the conductivity of the polymer and provide a lower energy threshold for conductivity. Dopants may also help to specifically control the conductivity characteristics. There are many methods and materials useful in doping that may be known to those skilled in the art. Doping materials can include, but are not limited to chloride, polystyrene sulfonate (PSS), dodecylbenzenesulfonate, polystyrenesulfonate, naphthalene sulfonate, and lithium perchlorate.

The conductive polymer section or sections may be formed in any shape such as, for example, circular, oval, triangular, square, pentagonal, or any other shape desired. In some embodiments, the conductive polymer section 9 preferably has a thickness of from 0.01 μm to 500 μm, and in other embodiments from 0.1 to 2.0 μm. The ability to vary the thickness of the conductive polymer section 9 can enable varying the charge storage capacitance (CSC) of the layer based on varying usage needs. For example, a thicker conductive polymer section 9 will allow a greater CSC accumulation on the electrode which may be more suitable for some applications. In embodiments, the CSC of the conductive polymer layer 9 ranges from 1 to 400 mC/cm².

High electrode capacitance, and therefore low electrode impedance, and a high degree of biocompatibility are useful for electrodes in biological/medical applications, particularly applications that involve long-term use such as, for example, on a tissue stimulator having an exhaustible energy source and which therefore must contribute to the minimal energy consumption.

Still referring to FIG. 1 and FIG. 2, the dry adhesive section 11 comprises a plurality of fibrillar microstructures configured to provide adhesive forces between the electrode and the wearer to which it is attached. The plurality of fibrillar microstructures of the dry adhesive section 11 of the top layer 3 can reversibly adhere to tissue thereby reducing or eliminating the need to use a wet adhesive, such as conventional biocompatible glues known in the art for electrode attachment. The dry adhesive section 11 is suitably flexible to conform the tissue it contacts and to maintain the desired position of the conductive polymer section 9. In some uses, the electrode 1 can be adhered to the skin of a patient for diagnostic monitoring or treatment purposes. In such applications the electrode may be applied and removed multiple times for repositioning the electrode 1 without the need for applying repeatedly applying adhesive to the electrode or the skin.

The plurality of fibrillar microstructures of the dry adhesive section 11 of the top layer 3 can comprise materials that suitably exhibit van der Waals interactions with skin or other tissue. These include silicone rubber, polyurethane, polyester resin, polyimide, artificial rubber, epoxy resin, polydimethylsiloxane, polyurethane and ethylene glycol terephthalate or polymethyl methacrylate or any of their combinations. Polymers that are elastomers such as, for example, silicone are preferred. The term “elastomer” in the descriptions herein, refers to a material that changes properties in response to an applied force. Elastomers, in various formulations respond to normal forces, compression, torque, or sheer stresses or forces. Some elastomers are also referred to as “rubber,” “polymer,” or “silicone.” Typically, but not always, an elastomer responds to an applied force with a physical deformation. Additionally, elastomers can be designed to change various properties such as impedance in response to applied force, stress, or torque. Elastomers can be configured to change properties when stressed in one dimension, or in multiple dimensions.

Elastomers can be formulated and produced with various properties that may be desirable for a given application, for example desired flexibility, stiffness (e.g., spring constant or dimensional change in response to pressure), conformability (e.g., ability to follow a curved or complex contour), thickness, color, or electrical or heat conductivity. Another property of an elastomer is “durometer,” which is its hardness or resistance to permanent deformation.

Silicone or other elastomers used for the adhesive microstructure can include nanostructures that have a surface pattern which enables the silicone to strongly adhere to a tissue and be retained even during movement of a patient. Silicone offers several desired properties including biocompatibility, resistant to bacteria, and easy, gentle removal and reapplication to tissue.

Fibrillar microstructures having different physical characteristics, such as in shape, size, and/or volume, can comprise different adhesive properties. For example, the plurality of fibrillar microstructures may comprise a plurality of structures selected from the group consisting of a wedge, a conical structure, cylindrical structure, a trapezoidal structure, a mushroom shape, and a cubical structure.

In some aspects, physical characteristics such as a shape, size, or volume of fibrillar microstructures in a dry adhesive may affect the degree of van der Waals interactions between the fibrillar microstructures and a contact surface to enhance or diminish overall adhesive performance. If not originally formed, the plurality of fibrillar microstructures may be post-treated to change one or more physical characteristics to improve adhesive performance. For example, the plurality of fibrillar microstructures may be post-treated to comprise mushroom-like flaps at their tips to interface with the contact surface. An increased contact area at the interface can increase attractive forces (e.g., van der Waals interactions).

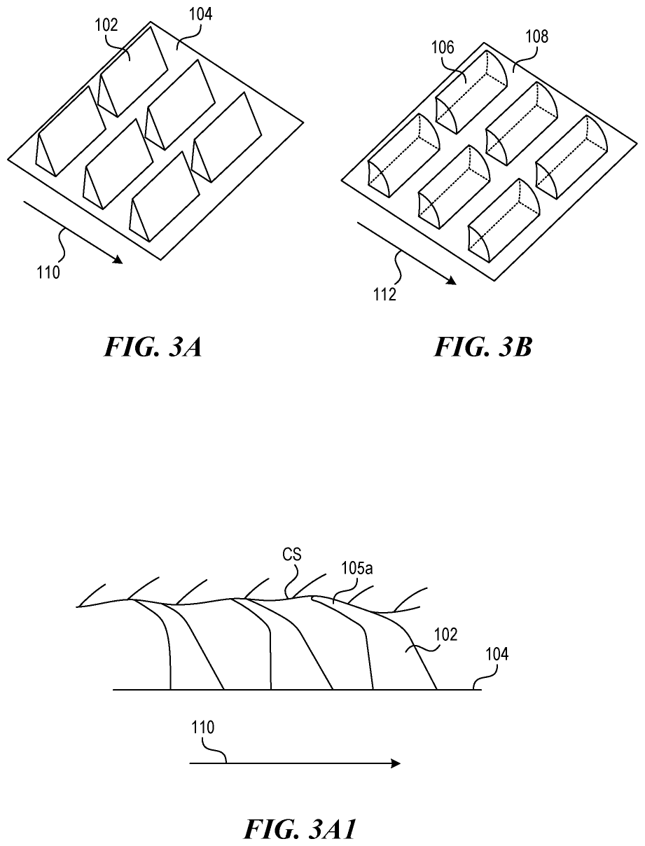

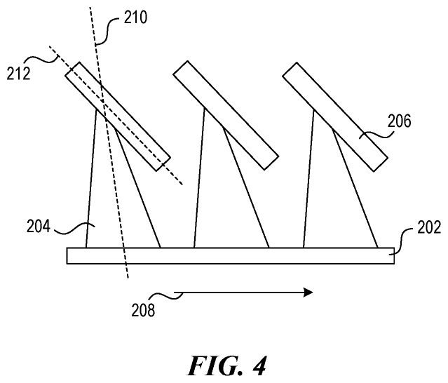

FIG. 3A, which is not drawn to scale, illustrates a perspective view of an exemplary embodiment of a plurality of fibrillar microstructures (in an unloaded state) that may be found on the dry adhesive section 11 of the electrode 1 disclosed herein. A plurality of microstructures 102 may populate at least a portion of a surface 104 of the top layer 3 in the dry adhesive section 11. The surface 104 can represent a sample portion of a larger surface intended for adhesion. A microstructure “stalk” may comprise two ends, a first end 105a at in the surface 104 of the top layer 3 and a second end 105b, such as a tip of the stalk, extending longitudinally away from the top layer 3. The tip of 105b a fibrillar microstructure stalk may be pointed. Alternatively, the tip 105b of a fibrillar microstructure stalk may be flat, rounded, or comprise a more complex pattern. For example, the tip 105b of each of the plurality of stalks may have a shape selected from the group consisting of pointed, rounded inward, rounded outward, and flat, wherein each shape may have a diameter larger than a diameter of the stalk to which it is attached. Each of the fibrillar microstructures 102 may comprise substantially uniform geometric structures. For example, FIG. 3A shows an array of uniform wedge-like fibrillar microstructures wherein the cross-sectional front view of each fibrillar microstructure is triangular (see FIG. 4A) with a base 105a at on the surface 104 and a tip 105b extending longitudinally away from the surface 104. The wedge-like fibrillar microstructures 102 shown in FIG. 3A are expected to be easier to manufacture than some other shapes of fibrillar microstructures, and they work well in multiples directions of applied force and facilitate the attachment and release of an electrode. Alternatively, the microstructures 102 may comprise conical, cylindrical, cubical, trapezoidal, or other more complex geometric structures with similar or different cross-sectional shapes.

The fibrillar microstructures 102 can have micro-scale dimensions. For instance, a fibrillar microstructure can have a maximum dimension of less than about 300, 250, 200, 150, 140, 130, 120, 110, 100, 90, 80, 70, 60, 50, 45, 40, 35, 30, 25, 20, 15, 10, 9, 8, 7, 6, 5 μm. A maximum dimension of the microstructure may be a dimension of the microstructure (e.g., length, width, height, altitude, diameter, etc.) that is greater than the other dimensions of the microstructure. In one example, the wedge-like microstructures 102 can have dimensions of about 60 μm in height, 20 μm in width, and 200 μm in length. In some instances, each of the fibrillar microstructures 102 may be laid out on the surface 104 in an evenly spaced array or a grid-like pattern. For example, an edge of the base of each microstructure 102 may be separated from the closest edge of the base of a neighboring microstructure by a distance of about 20-40 μm. In other instances, each of the fibrillar microstructures 102 may be laid out in an arbitrary pattern with non-uniform gaps (e.g., a random or otherwise irregular pattern of gaps) between each microstructure.

Directional control, wherein a gripping surface comprising the fibrillar microstructures 102 is configured to adhere to a contact surface when a shear load is applied in a preferred direction 110 and detach when the shear load is relaxed, can be achieved by orienting each fibrillar microstructure 102 in substantially the same direction on the surface 104. The tip, or a characteristic axis (e.g., axis 210 in FIG. 4), of each microstructure can be configured to tilt away from the preferred direction 110 of shear load. The characteristic axis can be a longitudinal axis. In the unloaded state, as in FIG. 3A, the tips of the wedge-like fibrillar microstructures 102 allow only for minimal contact area between the microstructures and a contact surface, which allows for relatively low van der Waals interactions and therefore low adhesive performance. When a shear load is applied to the fibrillar microstructures 102 in the preferred direction 110, the fibrillar microstructures 102 can conform, or bend, against the contact surface, as in FIG. 3B (contact surface not shown), in a direction opposite the preferred direction 110 such that the contact area between the fibrillar microstructures and the contact surface significantly increases. This produces relatively high van der Waals interactions and therefore higher adhesive performance. When the shear load is relaxed, the fibrillar microstructures 102 can revert to the initial unloaded state, as in FIG. 3A. The fibrillar microstructures 102 may comprise a compliant material (e.g., elastomers) that can withstand repeated structural conformations between the unloaded state and the loaded state. The materials comprising the microstructures will be discussed further below.

FIG. 3B illustrates a perspective view of another exemplary fibrillar microstructures on a surface of the top layer 3 in a loaded state. FIG. 3B is not drawn to scale. A plurality of fibrillar microstructures 106 may populate at least a portion of a surface 108 of an electrode as disclosed herein or any other surface intended for adhesion, such as the dry adhesion section 11. The fibrillar microstructures 106 can have a first end 107a at the surface 108, a second end 107b defining a tip, and a curved surface 107c between the first end 107a and the second end 107b. In the loaded state, as described above, a shear load is applied in a preferred direction 112 which bends the fibrillar microstructures 106 against a contact surface (not shown in FIG. 3B) in a direction opposite the preferred direction 112, increasing the real contact area between the fibrillar microstructures 106 and the contact surface. The wedge-like fibrillar microstructures 106 have an extended length along the curved surface 107c between the first end 107a and the second end 107b that may provide increased contact area with the skin of the user. In some instances, the surfaces 104 and 108 can be the same surface, the preferred directions 110 and 112 can be the same direction, and the fibrillar microstructures 102 and fibrillar microstructures 106 may represent the same microstructures in an unloaded state and a loaded state, respectively.

FIG. 4 illustrates one embodiment of a tip that may be originally formed or subsequently modified. FIG. 4 shows wedge-shaped fibrillar microstructures 204 comprising post-treatment tips 206 that resemble mushroom-like flaps. The longitudinal axis 212 of each post-treatment tip can tilt away from the direction 208 at a larger angle than the longitudinal axis 210 of the fibrillar microstructures 204. The post-treatment tips 206 provide a larger contact area compared to microstructures 102 and 106 that can produce greater attractive forces (e.g., higher Van der Waals interactions) and improve the adhesive performance of the dry adhesive surface.

The dry adhesive performance of these fibrillar microstructures can be significantly enhanced through a post-treatment process that reconfigures the respective tips of each fibrillar microstructure to add a mushroom-like flap or other shape to the tip of each fiber. During a loaded state of the microstructures, the modified tip may provide increased contact area between the microstructures and the contact surface. One procedure for making such modified tips is a post treatment process found, for example, in U.S. Pat. No. 10,363,668, the disclosure of which is incorporated herein by reference.

In some embodiments, the fibrillar microstructures 102, 106 and 204 of the dry adhesive section 11 of the top layer 3 are free of conductive particles. In other embodiments, the fibrillar microstructures 102, 106 and 204 may include and/or be at least partially coated with a conductive material (e.g., conductive particles).

The middle layer 5 of the electrode 1 functions in part to provide mechanical properties and flexibility to the electrode as well as to electrically insulate the conductive polymer section 9 from the bottom layer 7. The middle layer 5 may also provide a water-resistant or water-proof layer. The middle layer 5 may comprise any non-conductive material (e.g., a polymer), such as the elastomers detailed above from which the dry adhesive section is made. These include silicone rubber, polyurethane, polyester resin, polyimide, artificial rubber, epoxy resin, polydimethylsiloxane, polyurethane and ethylene glycol terephthalate or polymethyl methacrylate or any of their combinations. In some embodiments, the middle layer 5 can have a thickness of from about 0.1 mm to about 10 mm, and preferably from about 0.5 to about 3 mm.

The bottom layer 7 can comprise a conductive material (e.g., a polymer), such as the same conductive polymer as the conductive polymer section 9 on the top layer 3 as described above. These include polynaphthalene, polythiophene, polyethylene oxide, and polyethyldioxythiophene (PEDOT). Other classes of conductive polymers suitable for the bottom layer 7 include polyacetylenes, conductive polypyrrole polystyrene sulfonate, polythiophenes (PT), and polyanilines. Conductive polymers may also include EHPT (poly(3-(2-ethylhexyl)thiophene) ionomers (e.g., NAFION®), poly(3,4 ethylene dioxythiophene) (PEDOT) and PEDOT polystyrene sulfonate (PSS/PEDOT), polyacrylamide, and polyvinylpyrrolidone.

In some embodiments, the bottom layer 7 has a thickness of from about 0.01 μm to about 500 μm, and in other embodiments from about 0.1 μm to about 2.0 μm. Ultimately, the thickness of the bottom layer 7 as well as top layer 3 and conductive polymer section 9 will depend on the particular application.

The bottom layer 7 is electrically coupled with the conductive polymer section 9 of the top layer 3. Referring to FIG. 1 as an example, the bottom layer 7 is in electrical communication with the conductive polymer section 9 of top layer 3 by a via or pathway 13 through the non-conductive middle layer 5 such that the via 13 effectively acts as a wire connection between the two conductive regions. There may also be two or more vias arranged in any pattern between the conductive polymer section 9 and the bottom layer 7. The conductive polymer for conductive polymer section 9, bottom layer 7, and via 13 can be formed by a spray coating process or any other process for forming a conductive polymer layer as is known to those skilled in the art.

Additional embodiments may also include another non-conductive removable backing layer (not shown) on the bottom layer 7 to electrically insulate and protect the bottom layer 7 until use. The backing layer may comprise any of adhesive-containing paper or non-conductive polymer or other electrically insulating material. The removable backing layer may also be made from liners made of or coated with polyethylene, polypropylene, and fluorocarbons and silicone coated release papers or polyester films, for example.

In operation, the bottom layer 7 is in electrical connection with a terminal (not shown) via a conducting element (not shown) such as, for example, a wire, which electrically connects the electrode to a current delivery device (not shown). The current delivery device may contain electronics to sense various electrical signals of a particular body part such as, for example, the heart or the brain (e.g., an EEG), and/or produce current pulses for delivery to a body part such as, for example, the heart or brain via the connecting element. The current delivery or receiving device may contain electronics to sense various electrical signals from particular body part or tissue such as the heart or the brain and to also produce current pulses for delivery to a body part or tissue as desired.

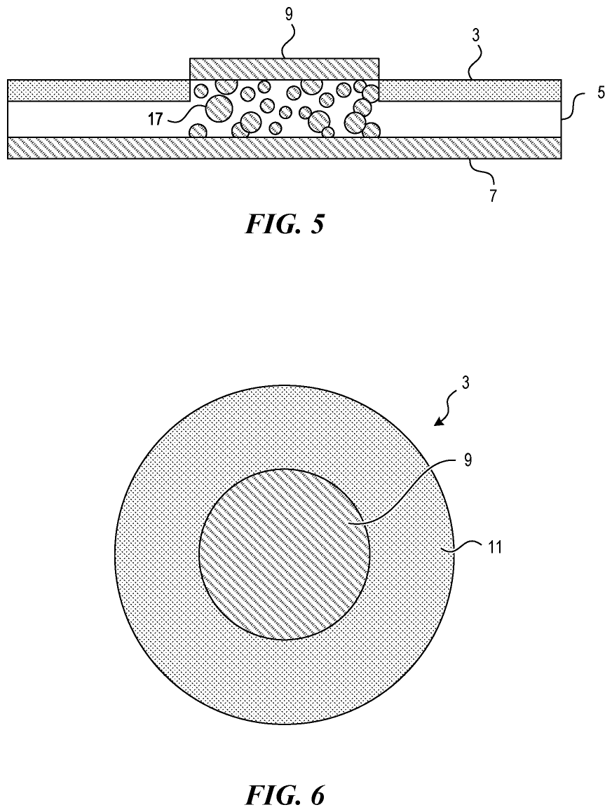

FIG. 5 and FIG. 6 illustrate another embodiment wherein the bottom layer 7 is in electrical communication with the conductive polymer section 9 of the top layer 3 by means of a via or pathway 17 through the non-conductive at least one middle layer 5 such that the via effectively acts as a wire connection between the two conductive regions. In this embodiment, the via 17 comprises a porous elastomer that is either coated with or impregnated with a conductive polymer that enables electrical signals to travel between the bottom layer 7 and the conductive polymer 9. Examples of non-electrically conductive elastomeric foams include silicone, polyurethane, polyethylene, polyacrylate, P(M)MA, ABS, PVDF, polyester, polyamide, polyimide, SEBS, PEEK, PPS, PEVA, PEN, polysulfone, and copolymers and mixtures thereof.

The porous elastomer such as, for example, silicone can be made by techniques known in the art. The porous silicone is coated or impregnated with a conductive polymer such as any of the conductive polymers detailed above for use in the conductive polymer section of the top layer or the bottom layer. For example, the porous elastomer can be an open polymer foam or a closed polymer foam. An open polymer foam can be coated using dip coating methods known in the art. An open polymer foam need not be electrically conducting since dipping the foam in a conductor can disperse conducting material throughout the polymer foam. Closed polymer foams can be coated with a conducting polymer using a printing or spraying method.

One aspect of several electrodes in accordance with the present technology is that they are free of hydrogels. Additionally, electrodes in accordance with the present disclosure may include additional features not illustrated herein, for example, adhesive layers bonding the various components of the electrode together, labeling, a mechanism for holding the electrical conductor in place and in electrical contact with the conductive element, and/or packaging. Components of electrodes in accordance with examples of the present disclosure may be formed from materials having certain desirable properties. Further, electrodes in accordance with the present disclosure may communicate wirelessly with circuitry.

Electrodes in accordance with the present disclosure may be substantially flat. For example, the electrodes may have a flat profile that is not noticeable or is minimally noticeable when attached to the subject, under the subject's clothes. The electrodes may also be substantially flexible. For example, the electrodes may conform to the contours of the subject's body during initial attachment to the subject and may conform to body positioning changes when the subject is in motion. The electrodes can also be substantially devoid of rigid components, such as hard snaps, connectors, and rigid plates. For example, the electrodes may be devoid of hard rigid substances that may cause uncomfortable pressure points when a subject with the electrodes attached to his/her body is in a prone, prostrate, supine, or lateral position with the electrodes pressed against an object, such as a bed, couch, medical examining table, clothes, or medical equipment.

Accordingly, the present disclosure includes the disclosure of a medical device comprising: a current delivery device; at least one electrode, wherein the at least one electrode comprises: a top layer comprising at least one conductive polymer section and at least one dry adhesive section, wherein the at least one dry adhesive section comprises a plurality of fibrillar microstructures; at least one middle layer comprising a non-conductive polymer; and a bottom layer comprising a conductive polymer, wherein the conductive polymer of the bottom layer is in electrical communication with the conductive polymer of the top layer through the at least one middle layer; and at least one connector element connecting the at least one electrode to the current delivery device.

The electrodes disclosed herein may also be incorporated into a smart garment, which may or may not serve as a medical device. Accordingly, the present disclosure includes the disclosure of a wearable garment comprising at least one electrode, wherein the at least one electrode comprises: a top layer comprising at least one conductive polymer section and at least one dry adhesive section, wherein the at least one dry adhesive section comprises a plurality of fibrillar microstructures; at least one middle layer comprising a non-conductive polymer; and a bottom layer comprising a conductive polymer, wherein the conductive polymer of the bottom layer is in electrical communication with the conductive polymer of the top layer through the at least one middle layer.

The garment incorporating at least one of the electrodes disclosed here could be any garment such as a shirt, a strap, a brace, a belt, and the like. Examples of wearable garments include those found in U.S. Pat. No. 9,545,514, the disclosure of which is incorporated herein by reference.

### Examples

The following description is a procedure to make the embodiment of FIG. 1 and FIG. 2.

In a first step, the substrate for the patch is manufactured including the fibrillar microstructures. There are several options to produce the part using established methods that are known to those skilled in the art. One first way would be the use of micro molding as described in Liu et al., Fabrication of High Aspect Ratio Microfiber Arrays that Mimic Gecko Foot Hairs, Bionic Engineering, Vol. 57, No. 4, 404-408 (February 2012), which is incorporated herein by reference, to create a disc of 30 mm diameter from silicone or other elastomers, for example, by using a selectively micropatterned tooling surface to create the fibrillar microstructures at chosen sites of the disc. Apart from this first option of producing singular samples in a batch-type process, there is an alternative in continuously producing microstructured roll material from silicone or other polymers by using hot embossing, for example, in a plate-to-plate, roll-to-plate, or roll-to-roll configuration. The thus prepared roll material is then used to prepare individual parts, for example, mechanically by means of stamping or contactless with laser or waterjet cutting.

In a second step, one or more vias are created as through-holes in the substrate from the top to the bottom surface. The diameter of each individual via should be comparably small, for example, 0.5 mm or less to make use of capillary forces that are required to suck in the coating ink into the cavity in a subsequent coating process step. In one example, five vias aligned on a cycle with a virtual diameter of 5 mm are created by using a tool with five needle-like pins or protrusions to perforate the substrate lying on a support with according recesses to accommodate the pins of the aforementioned tool. As an alternative, laser or waterjet cutting may also be used to create the vias.

In a third step, the fibrillar microstructures are masked prior to coating to prevent the gecko-feet structure from being coated along with the rest of the substrate. To do so, a release liner with one more aperture is used, effectively covering the microstructured dry adhesive areas while leaving the non-structured surface areas uncovered and exposed. The dry adhesive properties of the microstructured area help to keep the liner in place.

In a fourth step, the to-be-coated surface area needs to be activated to allow for a proper wetting. Depending on the substrate material, the procedure may vary, comprising both chemical and physical means to activate the substrate. For the former one, a chemical treatment with H₂O₂ is an option while physical treatments may encompass low pressure oxygen plasma exposure or UV treatment, for example for 2 min with a 172 nm radiation.

In the fifth step, the previously masked and properly primed surface is coated with Tecticoat™ conductive polymer composition using a spraying gun “Krautzberger Handspritzapparat Mignon 4S” with 1.5 bar pressure, 9 cm working distance, and 2 sec spraying time. The respective liquid film will have a thickness on the order of 20 μm to 30 μm.

In the sixth step, the parts are dried for 10 min at 100° C. in a box oven so that the volatile solvents are evaporated, and the film is cured to form a solidified layer of about 0.3 μm to 1.0 μm thickness. The fourth to sixth steps are repeated for the bottom layer, eventually forming a conductive layer on top and on bottom, both of which are connected through the via that is likewise coated and filled.

In the last step, the liner is removed, and the electrode may be packed for storage and later use.

In the foregoing detailed description, the invention has been described with reference to specific embodiments. However, it will be appreciated that various modifications and changes can be made without departing from the scope of the invention as set forth in the appended claims.

### CLAUSES

The present technology is illustrated, for example, according to various aspects described below. Various examples of aspects of the present technology are described as numbered clauses (1, 2, 3, etc.) for convenience. These are provided as examples and do not limit the present technology. It is noted that any of the dependent clauses may be combined in any combination, and placed into a respective independent clause. The other clauses can be presented in a similar manner.
1. An electrode comprising:
   a top layer comprising at least one conductive polymer section and at least one dry adhesive section, wherein the at least one dry adhesive section comprises fibrillar microstructures;
   at least one middle layer comprising a non-conductive material; and
   a bottom layer comprising a conductive material, wherein the conductive material of the bottom layer is in electrical communication with the conductive polymer of the top layer through the middle layer.
2. The electrode of clause 1 wherein the conductive material of the bottom layer comprises a conductive polymer, and the conductive polymer of the conductive polymer section of the top layer is the same as the conductive polymer of the bottom layer.
3. The electrode of any of clauses 1-2 wherein the conductive polymer comprises poly(3,4-ethylenedioxythiophene) (PEDOT).
4. The electrode of any of clauses 1-3 wherein the fibrillar microstructures comprise an elastomer.
5. The electrode of any of clauses 1-4 wherein the elastomer comprises silicone.
6. The electrode of any of clauses 1-5, further comprising a via through the middle layer electrically coupling the conductive material of the bottom layer with the conductive polymer of the top layer.
7. The electrode of clause 6 wherein the via comprises conductive polymer.
8. The electrode of clause 6 wherein the via comprises a porous elastomer, and wherein the porous elastomer is impregnated with and/or at least partially coated by a conductive polymer.
9. The electrode of clause 8 wherein the porous elastomer is porous silicone and the conductive polymer comprises PEDOT.
10. The electrode of any of clauses 1-9 wherein the non-conductive material of the middle layer comprises an elastomer selected from the group consisting of silicone rubber, polyurethane, polyester resin, polyimide, artificial rubber, epoxy resin, polydimethylsiloxane, polyurethane, ethylene glycol terephthalate, polymethyl methacrylate, and combinations thereof.
11. The electrode of any of clauses 1-10 wherein the fibrillar microstructures comprise a plurality of structures selected from the group consisting of a wedge, a conical structure, cylindrical structure, a trapezoidal structure, a mushroom structure, and a cubical structure, wherein each structure has a first end rooted in the top layer and a second end extending longitudinally away from the top layer.
12. The electrode of clause 11 wherein the fibrillar microstructures comprise a plurality of wedges wherein each wedge has a first end rooted in the top layer and a second end extending longitudinally away from the top layer.
13. The electrode of any of clauses 1-12 wherein the fibrillar microstructures comprise a plurality of stalks wherein each stalk has a first end rooted in the top layer and a second end extending longitudinally away from the top layer ending in a tip.
14. The electrode of clause 13 wherein the tip of each stalk has a shape selected from the group consisting of pointed, rounded inward, rounded outward, and flat, wherein each shape may have a diameter larger than a diameter of the stalk to which it is attached.
15. The electrode of any of clauses 1-14 further comprising a backing layer in contact with the top layer.
16. The electrode of any of clauses 1-15 wherein the conductive polymer section is substantially circular.
17. The electrode of any of clauses 1-16 wherein the conductive polymer section is surrounded by the dry adhesive section.
18. A medical device comprising:
   a current delivery or receiving device;
   at least one electrode, wherein the at least one electrode comprises:
      a top layer comprising at least one conductive polymer section and at least one dry adhesive section, wherein the dry adhesive section comprises fibrillar microstructures;
      at least one middle layer comprising a non-conductive material; and
      a bottom layer comprising a conductive material, wherein the conductive material of the bottom layer is in electrical communication with the conductive polymer of the top layer through the middle layer; and
   at least one connector element connecting the electrode to the current delivery or receiving device.
19. The medical device of clause 18 wherein the conductive material of the bottom layer comprises a conductive polymer, and the conductive polymer of the conductive polymer section of the top layer is the same as the conductive polymer of the bottom layer.
20. The medical device of any of clauses 18-19 wherein the conductive polymer comprises poly(3,4-ethylenedioxythiophene) (PEDOT).
21. The medical device of any of clauses 18-20 wherein the fibrillar microstructures comprise an elastomer.
22. The medical device of clause 21 wherein the elastomer comprises silicone.
23. The medical device of any of clauses 18-22, further comprising an electrically conductive via through the middle layer electrically coupling the conductive material of the bottom layer with the conductive polymer section of the top layer.
24. The electrode of clause 23 wherein the via comprises a conductive polymer.
25. The electrode of clause 23 wherein the via comprises a porous elastomer, and wherein the porous elastomer is coated with and/or impregnated with a conductive polymer.
26. The electrode of clause 25 wherein the porous elastomer is porous silicone and the conductive polymer comprises PDOT.
27. The medical device of any of clauses 18-26 wherein the non-conductive polymer of the middle layer comprises an elastomer selected from the group consisting of silicone rubber, polyurethane, polyester resin, polyimide, artificial rubber, epoxy resin, polydimethylsiloxane, polyurethane, ethylene glycol terephthalate, polymethyl methacrylate, and combinations thereof.
28. The medical device of clause 27 wherein the elastomer comprises silicone.
29. The medical device of any of clauses 18-28 wherein the fibrillar microstructures comprise a plurality of structures selected from the group consisting of a wedge, a conical structure, cylindrical structure, a trapezoidal structure, and a cubical structure, wherein each structure has a first end rooted in the top layer and a second end extending longitudinally away from the top layer.
30. The medical device of clause 29 wherein the fibrillar microstructures comprise a plurality of wedges wherein each wedge has a first end rooted in the top layer and a second end extending longitudinally away from the top layer.
31. The medical device of any of clauses 18-30 wherein the fibrillar microstructures comprise a plurality of stalks wherein each stalk has a first end rooted in the top layer and a second end extending longitudinally away from the top layer ending in a tip.
32. The medical device of clause 31 wherein the tip of each stalk has a shape selected from the group consisting of pointed, rounded inward, rounded outward, and flat, wherein each shape may have a diameter larger that a diameter of the stalk to which it is attached.
33. The medical device of any of clauses 18-32 further comprising a backing layer in contact with the top layer.
34. The medical device of any of clauses 18-33 wherein the conductive polymer section is substantially circular.
35. The medical device of any of clauses 18-34 wherein the conductive polymer section is surrounded by the dry adhesive section.
36. A wearable device comprising:
   a substrate configured to be worn by a user; and
   at least one electrode comprising—
   a top layer comprising at least one conductive polymer section and at least one dry adhesive section, wherein the at least one dry adhesive section comprises fibrillar microstructures;
   at least one middle layer comprising a non-conductive material; and
   a bottom layer comprising a conductive material, wherein the conductive polymer of the bottom layer is in electrical communication with the conductive polymer of the top layer.
37. The wearable device of clause 36 wherein the conductive material of the bottom layer comprises a conductive polymer, and the conductive polymer of the conductive polymer section of the top layer is the same as the conductive polymer of the bottom layer.
38. The wearable device of any of clauses 36-37 wherein the conductive polymer comprises poly(3,4-ethylenedioxythiophene) (PEDOT).
39. The wearable device of any of clauses 36-38 wherein the fibrillar microstructures comprise an elastomer.
40. The wearable device of clause 39 wherein the elastomer comprises silicone.
41. The wearable device of any of clauses 36-40, further comprising an electrical via through the middle layer electrically coupling the conductive material of the bottom layer with the conductive polymer of the top layer.
42. The electrode of device 41 wherein the via comprises a conductive polymer.
43. The electrode of device 41 wherein the via comprises a porous elastomer coated with and/or impregnated with a conductive polymer.
44. The electrode of device 43 wherein the porous elastomer is porous silicon and the conductive polymer comprises PEDOT.
45. The wearable device of any of clauses 36-44 wherein the non-conductive material of the middle layer comprises an elastomer selected from the group consisting of silicone rubber, polyurethane, polyester resin, polyimide, artificial rubber, epoxy resin, polydimethylsiloxane, polyurethane, ethylene glycol terephthalate, polymethyl methacrylate, and combinations thereof.
46. The wearable device of clause 45 wherein the elastomer comprises silicone.
47. The wearable device of any of clauses 36-46 wherein the fibrillar microstructures comprise a structures selected from the group consisting of a wedge, a conical structure, cylindrical structure, a trapezoidal structure, and a cubical structure, wherein each structure has a first end rooted in the top layer and a second end extending longitudinally away from the top layer.
48. The wearable device of clause 47 wherein the fibrillar microstructures comprise a wedges wherein each wedge has a first end rooted in the top layer and a second end extending longitudinally away from the top layer.
49. The wearable device of any of clauses 36-48 wherein the fibrillar microstructures comprise a plurality of stalks wherein each stalk has a first end rooted in the top layer and a second end extending longitudinally away from the top layer ending in a tip.
50. The wearable device of clause 49 wherein the tip of each stalk has a shape selected from the group consisting of pointed, rounded inward, rounded outward, and flat, wherein each shape may have a diameter larger that a diameter of the stalk to which it is attached.
51. The wearable device of any of clauses 36-50 further comprising a backing layer in contact with the top layer.
52. The wearable device of any of clauses 36-51 wherein the conductive polymer section is substantially circular.
53. The device of any of clauses 1-52 wherein the conductive polymer section is surrounded by the dry adhesive section.
54. The device of any of clauses 1-53 comprising an article of clothing.
55. The device of any of clauses 1-53 comprising a portable monitor configured to be attached to the user.
56. The device of any of clauses 1-53 comprising an earpiece configured to be attached the user such that the electrode is positioned in an ear canal of the user.
57. The use of an electrode according to clauses 1 to 56.

1. An electrode comprising:
a top layer comprising at least one conductive polymer section and at least one dry adhesive section, wherein the at least one dry adhesive section comprises fibrillar microstructures;
at least one middle layer comprising a non-conductive material; and
a bottom layer comprising a conductive material, wherein the conductive material of the bottom layer is in electrical communication with the conductive polymer of the top layer through the middle layer.

2. The electrode of claim 1 wherein the conductive material of the bottom layer comprises a conductive polymer, and the conductive polymer of the conductive polymer section of the top layer is the same as the conductive polymer of the bottom layer.

3. The electrode of claim 1 wherein the conductive polymer comprises poly(3,4-ethylenedioxythiophene) (PEDOT).

4. The electrode of claim 1 wherein the fibrillar microstructures comprise an elastomer.

5. The electrode of claim 4 wherein the elastomer comprises silicone.

6. The electrode of claim 1, further comprising a via through the middle layer electrically coupling the conductive material of the bottom layer with the conductive polymer of the top layer.

7. The electrode of claim 6 wherein the via comprises conductive polymer.

8. The electrode of claim 6 wherein the via comprises a porous elastomer, and wherein the porous elastomer is impregnated with and/or at least partially coated by a conductive polymer.

9. The electrode of claim 8 wherein the porous elastomer is porous silicone and the conductive polymer comprises PEDOT.

10. The electrode of claim 1 wherein the non-conductive material of the middle layer comprises an elastomer selected from the group consisting of silicone rubber, polyurethane, polyester resin, polyimide, artificial rubber, epoxy resin, polydimethylsiloxane, polyurethane, ethylene glycol terephthalate, polymethyl methacrylate, and combinations thereof.

11. The electrode of claim 1 wherein the fibrillar microstructures comprise a plurality of structures selected from the group consisting of a wedge, a conical structure, cylindrical structure, a trapezoidal structure, a mushroom structure, and a cubical structure, wherein each structure has a first end rooted in the top layer and a second end extending longitudinally away from the top layer.

12. The electrode of claim 11 wherein the fibrillar microstructures comprise a plurality of wedges wherein each wedge has a first end rooted in the top layer and a second end extending longitudinally away from the top layer.

13. The electrode of claim 1 wherein the fibrillar microstructures comprise a plurality of stalks wherein each stalk has a first end rooted in the top layer and a second end extending longitudinally away from the top layer ending in a tip.

14. The electrode of claim 13 wherein the tip of each stalk has a shape selected from the group consisting of pointed, rounded inward, rounded outward, and flat, wherein each shape may have a diameter larger than a diameter of the stalk to which it is attached.

15. The electrode of claim 1 further comprising a backing layer in contact with the top layer.

16. The electrode of claim 1 wherein the conductive polymer section is substantially circular.

17. The electrode of claim 1 wherein the conductive polymer section is surrounded by the dry adhesive section.

18. A medical device comprising:
a current delivery or receiving device;
at least one electrode, wherein the at least one electrode comprises:
a top layer comprising at least one conductive polymer section and at least one dry adhesive section, wherein the dry adhesive section comprises fibrillar microstructures;
at least one middle layer comprising a non-conductive material; and
a bottom layer comprising a conductive material, wherein the conductive material of the bottom layer is in electrical communication with the conductive polymer of the top layer through the middle layer; and
at least one connector element connecting the electrode to the current delivery or receiving device.

19. The medical device of claim 18 wherein the conductive material of the bottom layer comprises a conductive polymer, and the conductive polymer of the conductive polymer section of the top layer is the same as the conductive polymer of the bottom layer.

20. The medical device of claim 18 wherein the conductive polymer comprises poly(3,4-ethylenedioxythiophene) (PEDOT).

21. The medical device of claim 18 wherein the fibrillar microstructures comprise an elastomer.

22. The medical device of claim 21 wherein the elastomer comprises silicone.

23. The medical device of claim 18, further comprising an electrically conductive via through the middle layer electrically coupling the conductive material of the bottom layer with the conductive polymer section of the top layer.

24. The electrode of claim 23 wherein the via comprises a conductive polymer.

25. The electrode of claim 23 wherein the via comprises a porous elastomer, and wherein the porous elastomer is coated with and/or impregnated with a conductive polymer.

26. The electrode of claim 25 wherein the porous elastomer is porous silicone and the conductive polymer comprises PDOT.

27. The medical device of claim 18 wherein the non-conductive polymer of the middle layer comprises an elastomer selected from the group consisting of silicone rubber, polyurethane, polyester resin, polyimide, artificial rubber, epoxy resin, polydimethylsiloxane, polyurethane, ethylene glycol terephthalate, polymethyl methacrylate, and combinations thereof.

28. The medical device of claim 27 wherein the elastomer comprises silicone.

29. The medical device of claim 18 wherein the fibrillar microstructures comprise a plurality of structures selected from the group consisting of a wedge, a conical structure, cylindrical structure, a trapezoidal structure, and a cubical structure, wherein each structure has a first end rooted in the top layer and a second end extending longitudinally away from the top layer.

30. The medical device of claim 29 wherein the fibrillar microstructures comprise a plurality of wedges wherein each wedge has a first end rooted in the top layer and a second end extending longitudinally away from the top layer.

31. The medical device of claim 18 wherein the fibrillar microstructures comprise a plurality of stalks wherein each stalk has a first end rooted in the top layer and a second end extending longitudinally away from the top layer ending in a tip.

32. The medical device of claim 31 wherein the tip of each stalk has a shape selected from the group consisting of pointed, rounded inward, rounded outward, and flat, wherein each shape may have a diameter larger that a diameter of the stalk to which it is attached.

33. The medical device of claim 18 further comprising a backing layer in contact with the top layer.

34. The medical device of claim 18 wherein the conductive polymer section is substantially circular.

35. The medical device of claim 18 wherein the conductive polymer section is surrounded by the dry adhesive section.

36. A wearable device comprising:
a substrate configured to be worn by a user; and
at least one electrode comprising—
a top layer comprising at least one conductive polymer section and at least one dry adhesive section, wherein the at least one dry adhesive section comprises fibrillar microstructures;
at least one middle layer comprising a non-conductive material; and
a bottom layer comprising a conductive material, wherein the conductive polymer of the bottom layer is in electrical communication with the conductive polymer of the top layer.

37. The wearable device of claim 36 wherein the conductive material of the bottom layer comprises a conductive polymer, and the conductive polymer of the conductive polymer section of the top layer is the same as the conductive polymer of the bottom layer.

38. The wearable device of claim 36 wherein the conductive polymer comprises poly(3,4-ethylenedioxythiophene) (PEDOT).

39. The wearable device of claim 36 wherein the fibrillar microstructures comprise an elastomer.

40. The wearable device of claim 39 wherein the elastomer comprises silicone.

41. The wearable device of claim 36, further comprising an electrical via through the middle layer electrically coupling the conductive material of the bottom layer with the conductive polymer of the top layer.

42. The electrode of device 41 wherein the via comprises a conductive polymer.

43. The electrode of device 41 wherein the via comprises a porous elastomer coated with and/or impregnated with a conductive polymer.

44. The electrode of device 43 wherein the porous elastomer is porous silicon and the conductive polymer comprises PEDOT.

45. The wearable device of claim 36 wherein the non-conductive material of the middle layer comprises an elastomer selected from the group consisting of silicone rubber, polyurethane, polyester resin, polyimide, artificial rubber, epoxy resin, polydimethylsiloxane, polyurethane, ethylene glycol terephthalate, polymethyl methacrylate, and combinations thereof.

46. The wearable device of claim 45 wherein the elastomer comprises silicone.

47. The wearable device of claim 36 wherein the fibrillar microstructures comprise a structures selected from the group consisting of a wedge, a conical structure, cylindrical structure, a trapezoidal structure, and a cubical structure, wherein each structure has a first end rooted in the top layer and a second end extending longitudinally away from the top layer.

48. The wearable device of claim 47 wherein the fibrillar microstructures comprise a wedges wherein each wedge has a first end rooted in the top layer and a second end extending longitudinally away from the top layer.

49. The wearable device of claim 36 wherein the fibrillar microstructures comprise a plurality of stalks wherein each stalk has a first end rooted in the top layer and a second end extending longitudinally away from the top layer ending in a tip.

50. The wearable device of claim 49 wherein the tip of each stalk has a shape selected from the group consisting of pointed, rounded inward, rounded outward, and flat, wherein each shape may have a diameter larger that a diameter of the stalk to which it is attached.

51. The wearable device of claim 36 further comprising a backing layer in contact with the top layer.

52. The wearable device of claim 36 wherein the conductive polymer section is substantially circular.

53. The wearable device of claim 36 wherein the conductive polymer section is surrounded by the dry adhesive section.

54. The wearable device of any of claims 1-53 wherein the substrate comprises an article of clothing.

55. The wearable device of any of claims 1-53 wherein the substrate comprises a portable monitor configured to be attached to the user.

56. The wearable device of any of claims 1-53 wherein the substrate comprises an earpiece configured to be attached the user such that the electrode is positioned in an ear canal of the user.

57. The use of an electrode according to claims 1 to 56.
